(19) United States
(12) Patent Application Publication
(10) Pub. No.: US 2010/0087486 A1
(43) Pub. Date: Apr. 8, 2010

(54) METHODS FOR USING TGF-B RECEPTOR INHIBITORS OR ACTIVIN-LIKE KINASE (ALK) 5 INHIBITORS A-83-01 AND SB-431542 TO TREAT EYE DISEASE AND WOUND HEALING CONDITIONS

(76) Inventors: Hiroshi Nakamura, Hudson, OH (US); Beatrice Y.J.T. Yue, Deerfield, IL (US)

Correspondence Address: BENESCH, FRIEDLANDER, COPLAN & ARONOFF LLP;ATTN: IP DEPARTMENT DOCKET CLERK, 200 PUBLIC SQUARE, SUITE 2300, CLEVELAND, OH, 44114-2378, US

(21) Appl. No.: 12/474,370

(22) Filed: May 29, 2009

Related U.S. Application Data
(60) Provisional application No. 61/057,461, filed on May 30, 2008.

(51) Int. Cl. A61K 31/444; A61K 31/4439; C07D 401/14; A61P 27/02

(52) U.S. Cl. 514/333; 514/338; 546/256; 546/274.1

(57) ABSTRACT

A pharmaceutical composition useful in the prevention of subconjunctival scarring that may occur after glaucoma filtration surgery comprising an effective amount of an activin receptor-like kinase 5 inhibitor. Also disclosed is a method of treating corneal haze and subconjunctival scarring that may develop after ocular surgery comprising applying an amount of a pharmaceutical composition including an activin receptor-like kinase 5 inhibitor.

This application claims priority to U.S. Provisional Patent No. 61/057,461, filed on May 30, 2008.

The development of this invention was supported by funding from the American Health Assistance Foundation, G2006-014 and the National Eye Institute, EY01792. The government has an interest in the invention.

### BACKGROUND

Ocular fibrotic wound response is a major cause of impaired vision and blindness, especially as a consequence of the surgical treatment for glaucoma. Glaucoma is a leading cause of blindness in the United States, and 2.5 million Americans and 65 million people worldwide were affected by the disease in 2000. Glaucoma is a disease characterized by damage to the optic nerve head, and neural and visual loss. One of the major risk factors of glaucoma is an elevated intraocular pressure (IOP) resulting from abnormalities in the aqueous humor outflow pathway. Glaucoma filtration surgery (GFS) is commonly performed when medication fails to control IOP adequately.

Excessive post-operation scarring often leads to failure of GFS. While the use of antimetabolites such as mitomycin-C (MMC) and 5-fluorouracil as conjunctival anti-scarring treatments benefits a number of patients, they do so by causing widespread cell death and are associated with severe and potentially blinding complications, such as hypotony maculopathy and infection. Therefore, other anti-scarring approaches have been investigated. In particular, transforming growth factor beta (TGF-β) and its pathway have emerged as a target for postoperative anti-scarring therapy.

### BRIEF DESCRIPTION OF DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

FIG. 1 is a side view of a human eye during glaucoma filtration surgery.

FIG. 2 is a graph showing the effect of ALK-5 inhibitor A-83-01 on the TGF-β signaling levels in cultured rabbit subconjunctival fibroblasts.

FIG. 3 is a graph showing the effect of ALK-5 inhibitor SB-431542 on the TGF-β signaling levels in cultured rabbit subconjunctival fibroblasts.

FIG. 4 is a Western blotting image showing the expression of connective tissue growth factor (CTGF) in cultured rabbit subconjunctival fibroblasts treated with ALK-5 inhibitors A-83-01 and SB-431542.

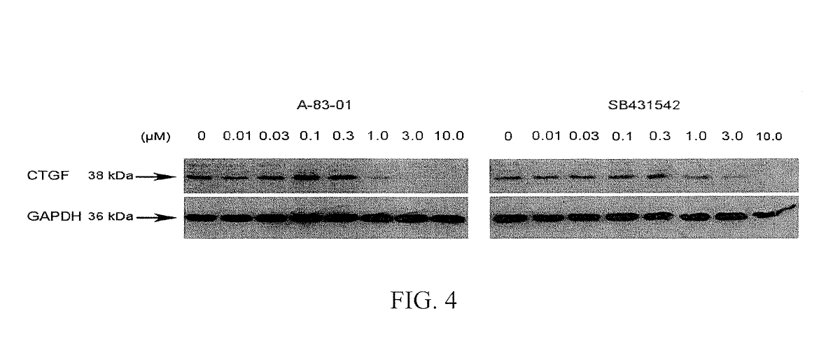

FIG. 5 is a Western blotting image showing the expression of fibronectin and α-smooth muscle actin (α-SMA) in cultured rabbit subconjunctival fibroblasts treated with ALK-5 inhibitors A-83-01 and SB-431542.

FIG. 6 is an immunocytofluorescense image showing the expression of CTGF, fibronectin, and α-SMA in cultured rabbit subconjunctival fibroblasts treated with ALK-5 inhibitors A-83-01 and SB-431542.

FIG. 7 is a phase contrast microscopy image showing the fibroblast morphology of cultured rabbit subconjunctival fibroblasts treated with ALK-5 inhibitors A-83-01 and SB-431542.

### DETAILED DESCRIPTION

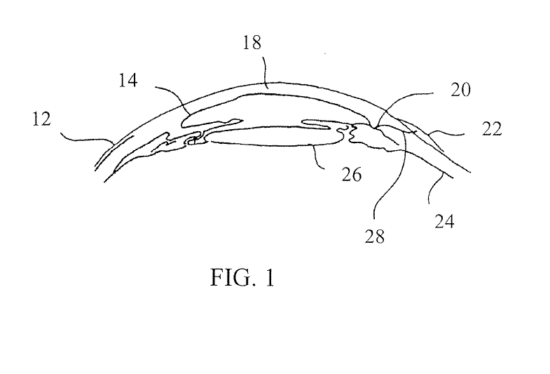

Disclosed herein are methods for preventing and treating eye disease and ocular scarring following glaucoma filtration surgery in a mammal. Preferably, the method may be used to treat human patients during or following glaucoma filtration surgery. In glaucoma filtration surgery (GFS), a new drainage site is created to facilitate drainage of fluid from the eye, thereby decreasing the intraocular pressure in the eye. As shown in FIG. 1, the human eye includes the conjunctiva 12, trabecular meshwork 14, iris 16, cornea 18, retina 24, and lens 26, among other components.

During GFS, instead of draining into the normal drainage site (the trabecular meshwork) 14 of the eye, the aqueous humor is drained into a new “space” that is created under the conjunctiva 12 of the eye. To do this, a small flap in the white of the eye is made. This is followed by the creation of a new drainage route 28 between the opening of the route 20 and a reservoir called a filtration bleb 22. The fluid in the anterior and posterior chamber, called the aqueous humor, can then drain into the bleb 22 via the new drainage route 28 and be absorbed into the vessels around the eye. The bleb 22 and/or the new drainage route 28 can scar and close preventing the aqueous humor from properly draining, called bleb failure.

Transforming growth factor-β (TGF-β) is a key mediator of wound healing responses. In the eye, TGF-β has been implicated in causing corneal haze after laser surgery and subconjunctival scarring following glaucoma filtration surgery. In addition, TGF-β upregulation is involved in proliferative vitreoretinopathy (PVR), which is a major cause for the failure of retinal detachment surgery.

The activin receptor-like kinase (ALK) 5 inhibitors may block the TGF-β signaling pathway, and thus, may be used to prevent corneal haze and scarring following ocular surgery, including GFS, vitreo-retinal surgeries, treatments of corneal trauma, and LASIK. Also, the use of the ALK-5 inhibitors may reduce the side effects associated with current anti-scarring medications, such as bleeding, infection, swelling, scarring, retinal detachment, a droopy eyelid, double vision, loss of vision, or even loss of the eye. Finally, topical application of ALK-5 inhibitors to the human eye may lower the intraocular pressure associated with glaucoma.

In one embodiment, one or more of the following compounds may be used. Manufacturer designation has been provided where available. The compounds are available from Sigma. P.O. Box 14508, St. Louis, Mo.

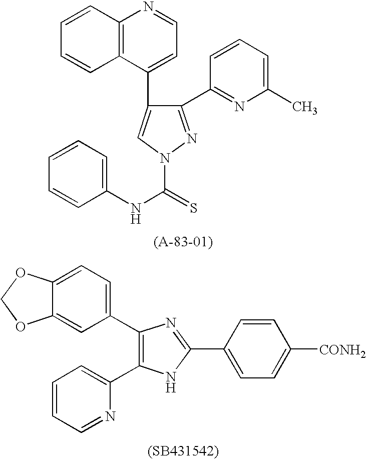

The above-described compositions may include ALK-5 inhibitors, and pharmaceutically acceptable salts thereof, that can be included in various types of pharmaceutical vehicles suitable for intraocular use, such as polymer carriers and carriers that are capable of forming gels upon administration. The vehicles are preferably aqueous, and are formulated to be chemically and physically compatible with ophthalmic tissues. For example, bioerodible (or biodegradable) gels or collagen inserts may be used to keep an effective concentration of the inhibitor in the bleb. The use of such gels or inserts has the advantage of providing a sustained release of the active components at the surgical site.

The compositions may include an effective amount of the ALK-5 inhibitor. Preferably, the compositions may include from about 0.3 to about 15 μM of the ALK-5 inhibitor, and more preferably from about 3 to about 10 μM of inhibitor. It should be appreciated by one of skill in the art that compositions including more than 15 μM may also be used.

As will be appreciated by those skilled in the art, the above-described compositions should be sterile and should not include any agents which will be toxic to sensitive intraocular tissues, particularly cornea/endothelial cells. The above described compositions can be formulated in accordance with techniques known to those skilled in the art.

The above described compositions can be applied to the surgical site by means of various techniques. For example, the compositions can be applied by means of a syringe during or immediately after surgery, preferably within 4 hours, or with a sustained release polymer that can be inserted into the eye on or around the surgical site. The compositions may be applied to the surgical site in a topical formulation following LASIK to prevent or reduce corneal haze.

### Examples

### In Vitro Cell Preparation with Inhibitors and TGF-β2

Sample fibroblasts were obtained from New Zealand white rabbit eyes. The fibroblasts were derived from the subconjunctival tissues isolated from the eyes of the subjects. The third to fifth passages of cells were maintained in 25 cm² flasks using 3 ml of medium composed of Eagle's minimal essential medium, 10% fetal bovine serum, 5% calf serum, essential and nonessential aminoacids, and antibiotics. When the cells reached confluence, they were trypsinized and passaged.

The fibroblast cultures in 6-well plates were pre-treated with 2 ml of medium including ALK-5 inhibitors at various concentrations, 0.03, 0.1, 0.03, 1.0, 3.0, and 10.0 μM, for one hour, and were additionally treated with

2 ng/ml of TGF-β2 (R&D Systems, Minneapolis, Minn.) for up to 72 hours. As shown in Table 1, samples 1-7 were prepared with ALK-5 inhibitor A-83-01 and samples 8-14 with ALK-5 inhibitor SB431542.

Samples 15 and 16 were prepared as controls. Sample 15 was not treated with an ALK-5 inhibitor or TGF-β2. Sample 16 was treated with 2 ng/ml of TGF-β, but not with an ALK-5 inhibitor. The samples were prepared as shown in Table 1, below.

TABLE 1

| | | Concentration Inhibitor | |
|---|---|---|---|
| Sample | ALK-5 | (μM) | TGF-β2 |
| # | Inhibitor | Pre-treat | (ng/ml) |
| 1 | A-83-01 | 0.01 | 2.0 |
| 2 | A-83-01 | 0.03 | 2.0 |
| 3 | A-83-01 | 0.1 | 2.0 |
| 4 | A-83-01 | 0.3 | 2.0 |
| 5 | A-83-01 | 1.0 | 2.0 |
| 6 | A-83-01 | 3.0 | 2.0 |
| 7 | A-83-01 | 10.0 | 2.0 |
| 8 | SB-431542 | 0.01 | 2.0 |
| 9 | SB-431542 | 0.03 | 2.0 |
| 10 | SB-431542 | 0.1 | 2.0 |
| 11 | SB-431542 | 0.3 | 2.0 |
| 12 | SB-431542 | 1.0 | 2.0 |
| 13 | SB-431542 | 3.0 | 2.0 |
| 14 | SB-431542 | 10.0 | 2.0 |
| 15 | N/A | 0.0 | 0.0 |
| 16 | N/A | 0.0 | 2.0 |

### Western Blotting for CTGF

Cells from the various samples were harvested following the treatment with the inhibitors and/or TGF-β, and Western blotting was performed to provide quantitative assessments. The conjunctival fibroblasts were lysed in a Triton lysis buffer. The total protein in the lysates was quantified using a Bradford protein assay. Equal amounts of protein (20 μg/lane) were resolved on a 10% sodium dodecyl sulfate (SDS)-polyacrylamide gel. The protein was then transferred to nitrocellulose membranes.

After blocking with 1% bovine serum albumin, the membranes were probed with polyclonal goat anti-CTGF (1:200, Santa Cruz Biotechnology, Santa Cruz, Calif., followed by HRP-conjugated donkey anti-goat IgG (1:1,000; Jackson ImmunoResearch, West Grove, Pa.). The TGF-β signal was detected by enhanced chemiluminescence (ECL) using SuperSignal from Pierce (Rockford, Ill.). Densitometry was then performed to measure the intensity of bands.

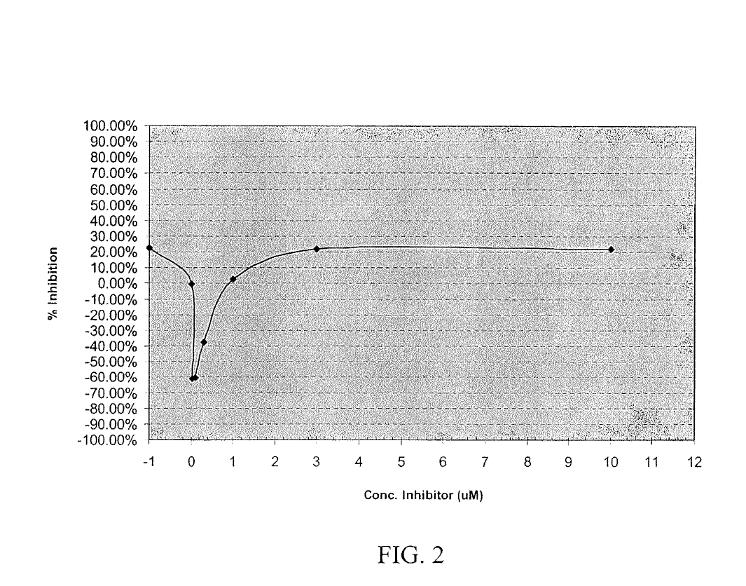

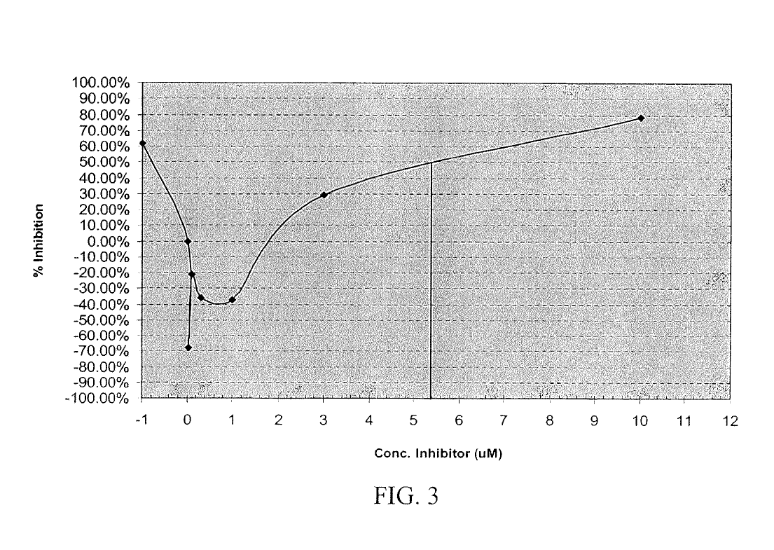

The densitometry showed reduced CTGF protein band intensities, i.e. 37-38 and 42-44 kDa, for the samples at concentrations above 1 μM, indicating diminished protein levels in the samples treated with the ALK-5 inhibitors. The membranes were also probed for the housekeeping gene, glyceraldehydes 3-phosphate dehydrogenase, as an internal standard. As shown in FIGS. 2-3, the half maximal inhibitory concentration (IC50) was calculated to evaluate effectiveness of each inhibitor in inhibiting TGF-β2 function. As the concentration of the inhibitors increased, the percentage TGF-β2 inhibited also increased. It is noted that the percentage of inhibition of the growth factor was dependent on the specific concentration of the respective inhibitors administered. Generally, the growth factor was inhibited to some extent by applying at least 1 μM of inhibitor to the cells. In some cases as much as 3 μM was required to provide inhibition of the signaling pathway. The control samples prepared without the inhibitors showed no inhibitory function of the TGF-β signaling pathway. It should be noted that in FIGS. 2-3, the “−1” demarcation on the graphs represents the expression percentage of the TGF-β downstream protein without ALK-5 inhibitors and TGF-β found when sample 15 was tested, and “0” demarcation represents the test data from a sample 16 tested without the respective ALK-5 inhibitor added, but with the TGF-β solution added.

Some samples prepared with low concentrations of the inhibitors actually showed an increase in the activity of the signaling pathway, leading to the conclusion that effective treatment with the inhibitors will be dependent on the specific inhibitor used and the concentration of the inhibitor applied to the surgical site. Moreover, it is desirable to maintain a constant concentration of the inhibitor on the surgical site over a prolonged period of time. Therefore, it may be desirable to apply the inhibitors with methods providing sustained release of the composition, such as with topical gels, polymer implants, and the like.

### Western Blotting for α-SMA, and Fibronectin

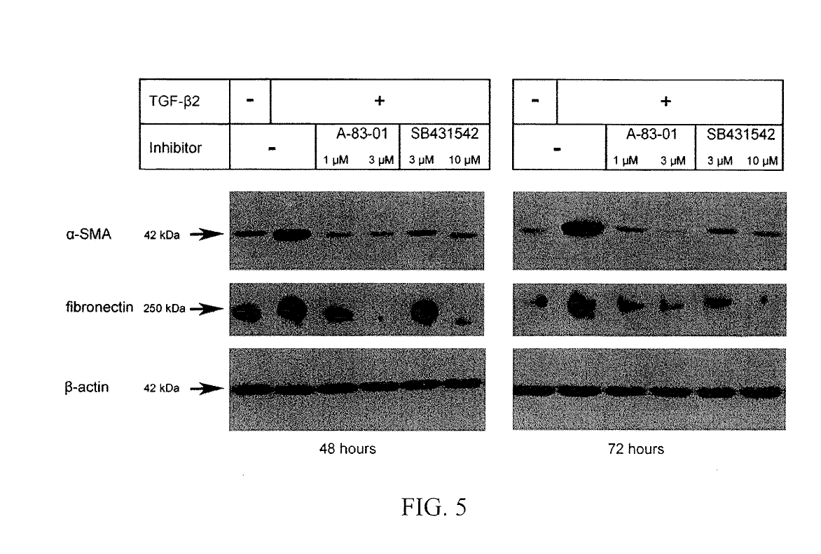

To test for fibronectin and α-SMA protein expression after the treatment with the respective inhibitors, rabbit subconjunctival fibroblasts, as prepared in samples 1-16, were incubated for 48 to 72 hours. As shown in FIG. 5, A-83-01 and SB-431542 effectively inhibited the protein expression induced by TGF-B2. Band intensity for both proteins was reduced in A-83-01 and SB-431542 treated samples compared with the bands of those samples not treated with the inhibitors. The reduction in protein levels is more pronounced with a higher concentration of the inhibitors. The blots were also probed for β-actin to control for equal protein loading.

The expression of α-SMA and fibronectin in fibroblasts that were treated with the A-83-01 and SB-431642, as detailed in Table 1, was measured to determine the extent to which the inhibitors blocked the TGF-β2 signaling pathway. Proteins in the cell lysates (20 μg/lane) were resolved on a 10% sodium dodecyl sulfate (SDS)-polyacrylamide gel. The proteins were transferred to nitrocellulose. After blocking with 1% bovine serum albumin, the membranes were probed with monoclonal mouse anti-α-SMA (1:9,000) followed by HRP-conjugated goat anti-mouse IgG (1:150,000; Jackson), or monoclonal mouse anti-fibronectin (1:1,000) followed by HRP-conjugated goat anti-mouse IgG (1:10,000). Signals were detected by enhanced chemiluminescense.

### Immunocytofluorescense Microscopy Imaging Techniques

In another example, subconjunctival fibroblasts were cultured on 8-well chamber slides. The samples were prepared as in samples 5-6 and 13-16 and incubated for 72 hours. After inhibitor treatment, the fibroblast cultures were fixed with 4% paraformaldehyde or with ice-cold methanol for Alexa Fluor or FITC staining, respectively. After permeabilization, the cells cultures were incubated with polyclonal goat anti-CTGF (1:50, Santa Cruz) followed by Alexa Fluor donkey anti-goat IgG (10 μg/mL, Invitrogen), monoclonal mouse anti-fibronectin (10 μg/mL, Invitrogen) or monoclonal mouse anti-α-SMA (1:400, Sigma) followed by FITC goat anti-mouse IgG (1:100, Jackson ImmunoResearch). The cell cultures were mounted with aqueous mounting media with DAPI and viewed by fluoresence microscopy.

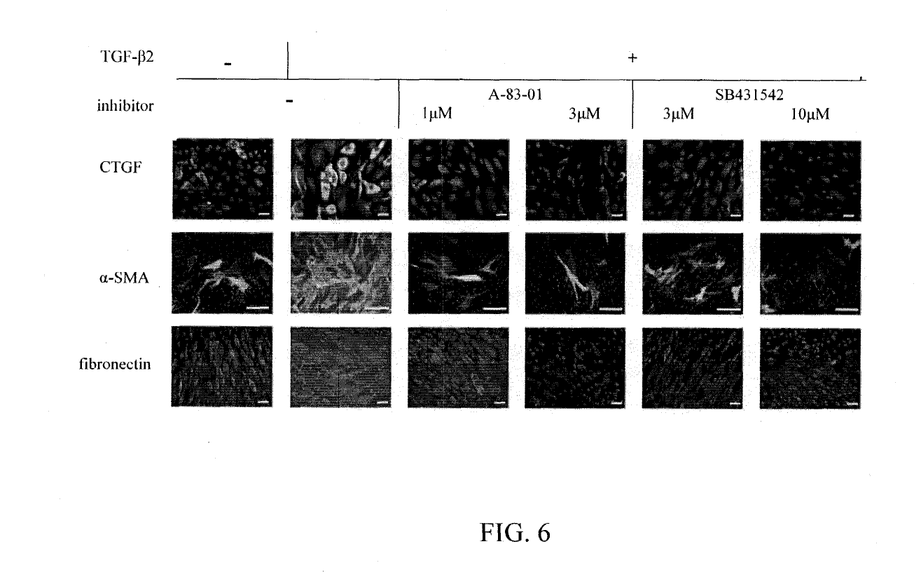

As shown in FIG. 6, CTGF, fibronectin, and α-SMA were visualized with FITC or Alexa Fluor labeling (green). Nuclei were stained with DAPI (blue). A dramatic increase in staining for CTGF, α-SMA, and fibronectin was observed following TGF-β2 incubation. The staining intensity of TGF-β2-induced proteins was greatly reduced when the cells were treated concomitantly with the A-83-01 or SB431542 inhibitors. No obvious cell death was observed in the samples treated with either inhibitor. Bar, 50 μM.

### Fibroblast Morphology

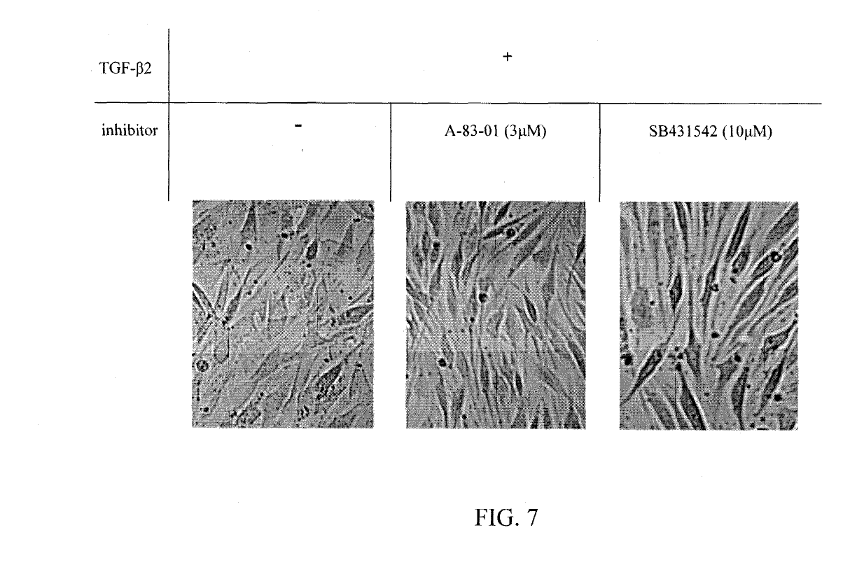

In another example, rabbit fibroblasts were prepared as in samples 6, 14, 15, and 16 except that 5 ng/ml of TGF-β2 was added to the samples instead of 2 ng/ml. The morphology of the cell cultures was visualized by phase contrast microscopy, as shown in FIG. 7. Myofibroblast-like appearance was observed in cells treated with TGF-β2. The TGF-β2-induced morphologic change seemed to be averted by addition of A-83-01 or SB431542. No obvious cell death was observed for the samples treated with the inhibitors.

The ALK inhibitors A-83-01 and SB-431542 effectively block TGF-β2 activity related to wound healing in cultured rabbit subconjunctival fibroblasts. No obvious cell toxicity was observed in the cell cultures prepared with either inhibitor. Thus, these inhibitors may be used as ocular anti-scarring agents, especially for glaucoma filtration surgery.

While example methods and compositions have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, devices, and so on, described herein. Additional advantages and modifications will readily appear to those skilled in the art. Therefore, the invention is not limited to the specific details, the representative apparatus, and illustrative examples shown and described. Thus, this application is intended to embrace alterations, modifications, and variations that fall within the scope of the appended claims. Furthermore, the preceding description is not meant to limit the scope of the invention. Rather the scope of the invention is to be determined by the appended claims and their equivalents.

What is claimed is:

1. A method of reducing the formation of scar tissue following ocular surgery and/or ocular injury, comprising:
applying to a post-surgical site or a post-injury site a composition comprising an effective amount of an activin receptor-like kinase 5 inhibitor selected from the group consisting of: 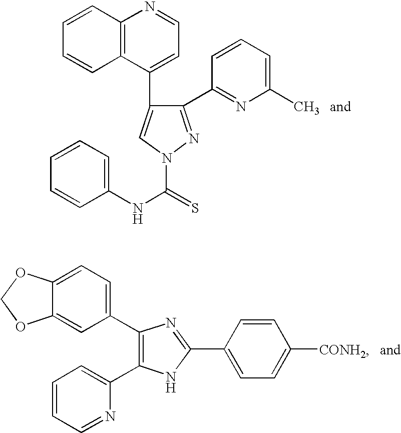
combinations thereof in an amount sufficient to inhibit the signaling pathway of transforming growth factor-β, and a pharmaceutically acceptable vehicle therefore.

2. The method of claim 1, wherein the activin receptor-like kinase 5 inhibitor is present in the composition in an amount from about 1.0 to about 10.0 μM.

3. The method of claim 1, wherein the activin receptor-like kinase 5 inhibitor is 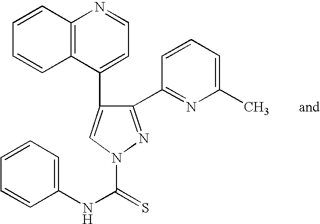
is present in the composition in an amount from about 1.0 to about 3.0 μM.

4. The method of claim 1, wherein the activin receptor-like kinase 5 inhibitor is 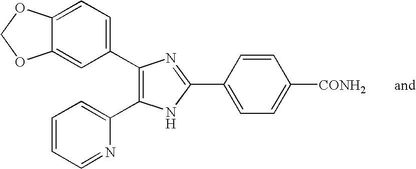
is present in the composition in an amount from about 1.0 to about 10.0 μM.

5. The method of claim 1, wherein the composition further includes a sustained release polymer carrier.

6. The method of claim 1, wherein the composition further includes a carrier medium capable of forming a gel upon administration to a surgical site on a patient's eye.

7. A method of reducing the formation of scar tissue following ocular surgery, comprising:
applying to the surgical site a composition comprising an effective amount of an activin receptor-like kinase 5 inhibitor selected from the group consisting of 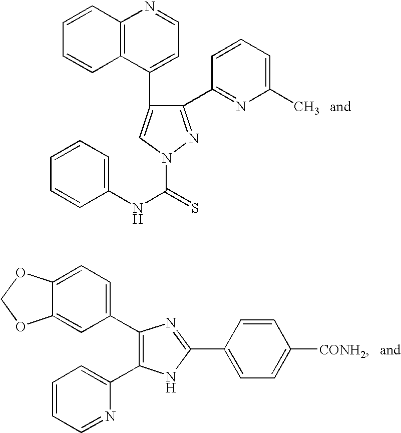
combinations thereof, in an amount sufficient to inhibit the signaling pathway of transforming growth factor-β, and a pharmaceutically acceptable vehicle therefore;
wherein the composition is applied in the form of a topical application to the surgical site following the surgery.

8. The method of claim 7, wherein the activin receptor-like kinase 5 inhibitor is present in the composition in an amount from about 1.0 to about 10.0 μM.

9. The method of claim 7, wherein the activin receptor-like kinase 5 inhibitor is 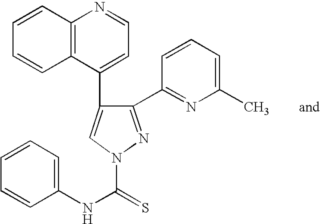
is present in the composition in an amount from about 1.0 to about 3.0 μM.

10. The method of claim 7, wherein the activin receptor-like kinase 5 inhibitor is 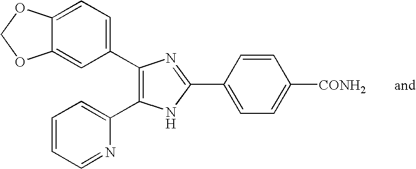
is present in the composition in an amount from about 1.0 to about 10.0 μM.

11. The method of claim 7, wherein the composition further includes a carrier medium capable of forming a gel upon administration to a surgical site on a patient's eye.

12. A pharmaceutical composition, wherein the composition is useful in preventing post-surgical ocular scarring and comprises an effective amount of an activin receptor-like kinase 5 inhibitor selected from the group consisting of: 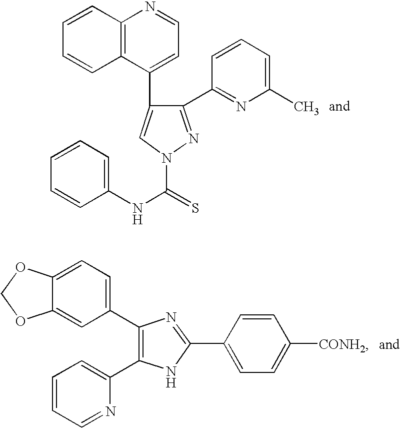
combinations thereof.

13. The composition of claim 12 comprising a pharmaceutically acceptable salt of the activin receptor-like kinase 5 inhibitor.

14. The composition of claim 12, wherein the activin receptor-like kinase 5 inhibitor is present in the composition in an amount from about 1.0 to about 10.0 μM.

15. The method of claim 12, wherein the activin receptor-like kinase 5 inhibitor is 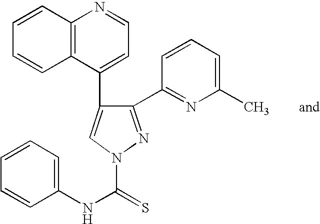
is present in the composition in an amount from about 1.0 to about 3.0 μM.

16. The method of claim 12, wherein the activin receptor-like kinase 5 inhibitor is 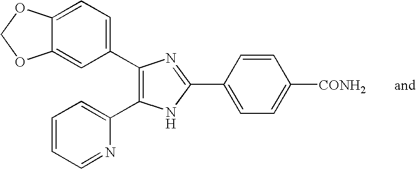
is present in the composition in an amount from about 1.0 to about 10.0 μM.

17. The composition of claim 12, wherein the composition further includes a polymer carrier.

18. The composition of claim 12, wherein the composition further includes a carrier medium capable of forming a gel upon administration to a surgical site on a patient's eye.
